(19) United States
(12) Patent Application Publication
(10) Pub. No.: US 2016/0317468 A1
(43) Pub. Date: Nov. 3, 2016

(54) USES OF CANNABIDIOL FOR TREATMENT OF INFANTILE SPASMS

(71) Applicants: Raman Sankar, Los Angeles, CA (US); Shaun A. Hussain, Los Angeles, CA (US)

(72) Inventors: Raman Sankar, Los Angeles, CA (US); Shaun A. Hussain, Los Angeles, CA (US)

(21) Appl. No.: 15/139,666

(22) Filed: Apr. 27, 2016

Related U.S. Application Data
(60) Provisional application No. 62/153,879, filed on Apr. 28, 2015.

(51) Int. Cl. A61K 31/05; A61K 31/352

(52) U.S. Cl. CPC A61K 31/05; A61K 31/352

(57) ABSTRACT

In some aspects, the invention relates to a method of treating or preventing infantile spasms in a subject, comprising administering to the subject a composition comprising cannabidiol (“CBD”). In certain preferred embodiments, the composition comprising CBD is substantially devoid of tetrahydrocannabinol (“THC”).

### CROSS-REFERENCE TO RELATED APPLICATIONS

This application claims the benefit of U.S. Provisional Application No. 62/153,879 filed on Apr. 28, 2015, the entire contents of which are incorporated by reference herein in their entirety.

### BACKGROUND

Infantile spasms are an often catastrophic disorder that affects children, particularly under the age of 24 months. For every 100,000 live births, about 20 to 35 children will develop infantile spasms. Infantile spasms are diagnosed following the observation of a distinct type of seizure (spasm), and are often accompanied by a psychomotor regression/arrest and hypsarrhythmic electroencephalogram (“EEG”) pattern. The spasms typically involve brief symmetrical contractions of the neck, trunk, and extremities, in clusters of 2 to 100 spasms. Early diagnosis and treatment are important for favorable outcomes, and current treatments comprise pharmaceutical interventions, surgery, and/or dietary interventions.

One of the most popular treatments for infantile spasms is intramuscular adrenocorticotropic hormone (“ACTH”) therapy. In at least one high-dose study, ACTH provided cessation of seizures and normal EEG patterns in 13 of the 15 patients (Baram, et al., PEDIATRICS, 97(3):375-79 (1996)). However, ACTH can cause severe side effects, such as hypertension, hyperglycemia, gastrointestinal bleeding, glycosuria, metabolic abnormalities, and fulminant infections secondary to immunosuppression, and many patients do not respond to ACTH.

Prednisone and prednisolone therapies are also effective in some patients. These treatments, however, may be less effective than ACTH, and they may also result in serious side effects similar to ACTH.

Vigabatrin is also used to treat infantile spasms. Vigabatrin, however, has been associated with intramyelinic edema in rodents and dogs and peripheral visual field defects. Thus, patients receiving vigabatrin must undergo routine vision screenings to minimize the risk of suffering progressive, permanent bilateral concentric peripheral vision field defects.

Additionally, surgery may be used to treat infantile spasms for subjects in which a surgeon can identify a zone of cortical abnormality (see Shields, et al., EPILEPSIA, 33(Suppl. 4):526-31 (1992)). Many subjects do not present with an obvious abnormality, however, and surgery involves inherent risks including anesthesia reactions, damage to healthy tissue, blood clot or bleeding in the brain, stroke, coma, and infection.

Outcomes for patients with infantile spasms are unpredictable, and many patients do not improve with any available treatment. Further, relapses frequently occur in subjects who initially respond to an intervention, and unresponsive patients face a multitude of complications including mental retardation and death. Accordingly, there is a need for new therapies to treat infantile spasms.

### SUMMARY

In some aspects, the invention relates to a method of treating or preventing infantile spasms in a subject, comprising administering to the subject a composition comprising cannabidiol (“CBD”).

In some aspects, the invention relates to a method of treating or preventing hypsarrhythmia in a subject, comprising administering to the subject a composition comprising cannabidiol.

In some aspects, the invention relates to a method of treating or preventing lightning attacks, nodding attacks, salaam attacks, or jackknife attacks in a subject, comprising administering to the subject a composition comprising cannabidiol.

In certain preferred embodiments, the composition comprising CBD is substantially devoid of tetrahydrocannabinol (“THC”).

### BRIEF DESCRIPTION OF THE FIGURES

FIG. 1 shows the design of a clinical trial assessing the safety and efficacy of a composition comprising cannabidiol in subjects who present with infantile spasms. Subjects are administered an increasing amount of cannabidiol per day during days 1 to 14, and subjects are monitored overnight by video-EEG on day 14. Cannabidiol treatment is optionally extended for subjects who respond to the therapy (Days 15-365). Cannabidiol therapy may be tapered after the overnight video-EEG (Days 15-28).

FIG. 2 shows the design of a clinical trial assessing the safety and efficacy of a composition comprising cannabidiol in subjects who present with infantile spasms. Subjects are administered an increasing amount of cannabidiol per day during days 1 to 14, and subjects are monitored overnight by video-EEG on day 14. Subjects' response to treatment is assessed, and the treatment may continue for several weeks depending on the subject's response to the composition during days 1 to 14.

### DETAILED DESCRIPTION

In some aspects, the invention relates to a method of treating or preventing infantile spasms in a subject, comprising administering to the subject a composition comprising cannabidiol (“CBD”). In some embodiments, the invention relates to a method of treating or preventing Lennox Gastaut syndrome, comprising administering to the subject a composition comprising cannabidiol. In some embodiments, the invention relates to a method of treating or preventing juvenile spasms, West syndrome, generalized flexion epilepsy, infantile epileptic encephalopathy, infantile myoclonic encephalopathy, jackknife convulsions, massive myoclonia, and/or salaam spasms in a subject, comprising administering to the subject a composition comprising cannabidiol. In some embodiments, the invention relates to a method of treating or preventing lightning attacks, nodding attacks, salaam attacks, and/or jackknife attacks in a subject, comprising administering to the subject a composition comprising cannabidiol. In some embodiments, the invention relates to a method of treating or preventing hypsarrhythmia in a subject, comprising administering to the subject a composition comprising cannabidiol. In certain preferred embodiments, the composition comprising CBD is substantially devoid of tetrahydrocannabinol (“THC”).

Cannabidiol, (−)-trans-2-p-mentha-1,8-dien-3-yl-5-pentylresorcinol, is one of many cannabinoids that naturally occur in cannabis. Synthetic cannabidiol has the same structure as naturally occurring cannabidiol (Formula I).

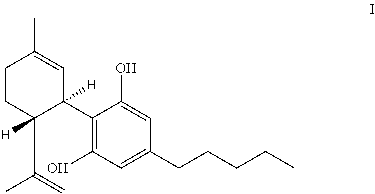 [SMILES: C=C(C)[C@@H]1CCC(C)=C[C@H]1c1c(O)cc(CCCCC)cc1O]

Commercially available cannabidiol usually contains Δ⁹-tetrahydrocannabinol (“THC”). In contrast, synthetic cannabidiol may be prepared without contaminating THC.

Cannabidiol may be synthesized by combining p-menthadienol and olivetol in toluene, dichloromethane, or hexane with a p-toluene sulfonic acid catalyst.

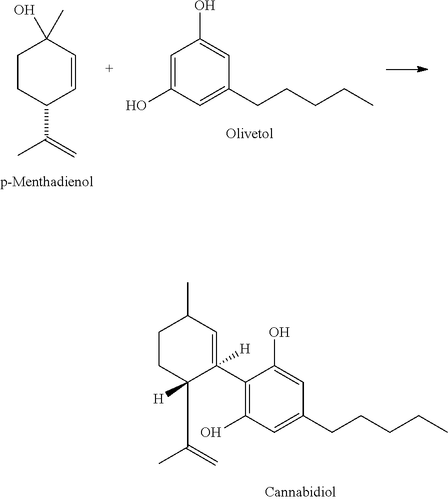

Synthesized cannabidiol may be purified to a purity of greater than 98%. The level of purity may be determined by chromatography. Preferably, the cannabidiol has a purity greater than 99%. More preferably, the cannabidiol has a purity greater than 99.5%.

In some embodiments, the composition comprises cannabidiol at a concentration of between about 1% and about 99.5% by weight (w/w). For example, the composition may comprise cannabidiol at a concentration of between about 2% and about 50% w/w, such as between about 4% and about 20% w/w, such as between about 5% and about 10% w/w. In some embodiments, the composition comprises about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% cannabidiol by weight.

In some embodiments, the composition is substantially devoid of tetrahydrocannabinol (“THC”). The amount of THC may be determined by chromatography. The composition may comprise THC at a concentration of less than 0.3% w/w, such as less than 0.2% w/w, or even less than 0.1% w/w. In some embodiments, the composition has a cannabidiol:THC ratio greater than 100:1, such as greater than 250:1, greater than 500:1, or greater than 1000:1.

In some embodiments, the composition is substantially devoid of cannabinoids selected from cannabichromene, cannabichromevarin, cannabicitran, cannabicyclol, cannabidivarin, cannabielsoin, cannabigerol, cannabigerol monomethyl ether, cannabigerovarin, cannabinol, cannabivarin, iso-tetrahydrocannabinol, and/or tetrahydrocannabivarin. The composition may comprise cannabinoids selected from cannabichromene, cannabichromevarin, cannabicitran, cannabicyclol, cannabidivarin, cannabielsoin, cannabigerol, cannabigerol monomethyl ether, cannabigerovarin, cannabinol, cannabivarin, iso-tetrahydrocannabinol, and/or tetrahydrocannabivarin at a concentration of less than 0.3% w/w, such as less than 0.2% w/w, or even less than 0.1% w/w. In some embodiments, the composition has a ratio of cannabidiol to other cannabinoids greater than 100:1, such as greater than 250:1, greater than 500:1, or greater than 1000:1.

In some embodiments, between about 1 mg/kg to about 200 mg/kg of cannabidiol is administered to the subject per day, such as between about 5 mg/kg to about 100 mg/kg, between about 10 mg/kg and about 80 mg/kg, between about 20 mg/kg and about 60 mg/kg, or between about 30 mg/kg and about 50 mg/kg. In some embodiments, about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg of cannabidiol is administered to the subject per day.

In some embodiments, between about 5 mg/kg to about 500 mg/kg of cannabidiol is administered to the subject per day, such as between about 10 mg/kg to about 400 mg/kg, between about 30 mg/kg and about 300 mg/kg, between about 50 mg/kg and about 200 mg/kg, or between about 75 mg/kg and about 150 mg/kg. In some embodiments, about 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, or 160 mg/kg of cannabidiol is administered to the subject per day. In some embodiments, 20 mg/kg of cannabidiol is administered to the subject per day. In some embodiments, after an initial treatment period, the method comprises increasing the dose of cannabidiol administered to the patient, e.g., from 20 mg/kg to 40 mg/kg per day, if the patient experienced a spasm during the initial treatment phase (or otherwise continues to show symptoms of the underlying disorder). The initial treatment phase may be at least about one week, about 1-4 weeks, at least about a month, or any other suitable time period for assessing the effectiveness of the treatment regimen on a particular patient.

In preferred embodiments, less than 10 mg of THC is administered to the subject per day. In more preferred embodiments, less than 5 mg of THC is administered to the subject per day, such as less than 4 mg of THC, less than 3 mg of THC, less than 2 mg of THC, or even less than 1 mg of THC. In some preferred embodiments, less than 900 μg of THC is administered to the subject per day, such as less than 800 μg of THC, less than 700 μg of THC, less than 600 μg of THC, less than 500 μg of THC, less than 400 μg of THC, less than 300 μg of THC, less than 200 μg of THC, or even less than 100 μg of THC.

In certain embodiments, less than 10 mg of cannabinoids other than cannabidiol (e.g., cannabichromene, cannabichromevarin, cannabicitran, cannabicyclol, cannabidivarin, cannabielsoin, cannabigerol, cannabigerol monomethyl ether, cannabigerovarin, cannabinol, cannabivarin, iso-tetrahydrocannabinol, tetrahydrocannabinol, and tetrahydrocannabivarin) are administered to the subject per day. In some embodiments, less than 5 mg of cannabinoids other than cannabidiol are administered to the subject per day, such as less than 4 mg, less than 3 mg, less than 2 mg, or even less than 1 mg of cannabinoids other than cannabidiol per day. In some preferred embodiments, less than 900 μg of cannabinoids other than cannabidiol are administered to the subject per day, such as less than 800 less than 700 μg, less than 600 μg, less than 500 μg, less than 400 μg, less than 300 μg, less than 200 μg, or even less than 100 μg.

The composition comprising cannabidiol may be administered to the subject 1, 2, 3, 4, 5, 6, 7, 8, 9, or 10 times per day. In some embodiments, the composition is administered 1, 2, 3, 4, or 5 times per day. In some embodiments, the composition is administered to the subject 2 or 3 times per day.

In some embodiments, the composition is administered orally.

In some embodiments, the subject is not responsive to adrenocorticotropic hormone therapy, prednisone or prednisolone therapy, and/or vigabatrin therapy. In some embodiments, the subject is responsive to adrenocorticotropic hormone therapy, prednisone or prednisolone therapy, and/or vigabatrin therapy.

The subject may be a human. In some embodiments the subject is under the age of 18 years. For example, the subject may be between the ages of 1 week and 18 years, such as between the ages of 1 week and 6 years, between the ages of 1 month and 48 months, or between the ages of 6 months and 36 months. In some embodiments, the subject is less than 24 months old.

The composition may be formulated for oral administration.

The composition may be administered to the subject for any suitable period of time. The subject may be administered the composition from about 1 to 20 weeks. The subject may be administered the composition for 1, 2, 3, 4, 5, 6, 7, 8, 9, 10, 11, 12, 13, 14, 15, 16, 17, 18, 19, or 20 weeks. In some embodiments, the composition comprising cannabidiol is administered to the subject for a period of about 12 weeks.

A formulation of the present invention may comprise a pharmaceutically acceptable sweetener, such as sucralose, sucrose, xylitol, sorbitol, or fructose. The formulation may comprise a sweetener from about 0.01% to about 3.0% w/w, such as from about 0.01% to about 0.10% w/w, from about 0.02% to about 0.08% w/w, or from about 0.04% to about 0.06% w/w.

In some embodiments, the pH of a formulation is between 3 and 9, such as between 4 and 8, or such as between 6 and 7.

In some embodiments, a formulation comprises a pH modifier. The pH modifier may be used to adjust the pH of the formulations. The pH modifier may be hydrochloric acid, citric acid, sodium citrate, fumaric acid, acetic acid, sodium hydroxide, sodium bicarbonate, sodium carbonate, ammonium carbonate, sodium phosphate, potassium phosphate, or a combination thereof. In some embodiments, the pH modifier is sodium hydroxide.

In some embodiments, a formulation comprises a preservative selected from benzyl alcohol, benzoic acid, sodium benzoate, sorbic acid, methyl paraben, and propyl paraben, or a combination thereof. A formulation may comprise a preservative at a concentration from about 0.01% to about 1.0% w/w, such as from about 0.02% to about 0.06% w/w, or from about 0.03% to about 0.05% w/w.

A formulation of the present invention may comprise a stabilizing agent or antioxidant. For example, a formulation may comprise a stabilizing agent or antioxidant selected from butylated hydroxyltoluene, butylated hydroxyl anisole, alpha-tocopherol, ascorbyl palmitate, ascorbic acid, sodium ascorbate, ethylenediamine tetra-acetic acid (EDTA), cysteine hydrochloride, citric acid, sodium citrate, sodium bisulfate, sodium metabisulfite, lecithin, propyl gallate, sodium sulfate, and/or combinations thereof. A formulation may comprise alpha-tocopherol and/or ascorbyl palmitate. A formulation may comprise an antioxidant or stabilizing agent at a concentration from about 0.01% to about 0.50% w/w of the formulation, such as from about 0.03 to about 0.30% w/w, or from about 0.04 to about 0.20% w/w.

Water may be used in the present formulations, e.g., as a solvent. If water is used, the formulations may comprise from about 0.01% to about 50% w/w water, such as from about 10% to about 40%, or about 20% to about 40%, or about 25% to about 35% w/w water. In some embodiments, the formulations comprise about 28% to about 32% w/w water.

Cosolvents may be used in the formulations of the present invention. As used herein, a “cosolvent” is a solvent other than water. A cosolvent may provide the proper formulation characteristics suitable for oral administration. When at least one cosolvent is used in a formulation, the formulation may comprise from about 0.01% to about 97% w/w of the cosolvent, such as from about 0.5 to about 90% w/w, from about 1% to about 80% w/w, or from about 30 to about 50% w/w.

A formulation of the invention may comprise at least one pharmaceutically acceptable alcohol, e.g., as a cosolvent, such as a lower alkyl (C₁-C₄) alcohol. In certain preferred embodiments, ethanol is the alcohol. A formulation may comprise an alcohol (e.g., ethanol) at a concentration of from about 0.5% to about 60% w/w, such as from about 10% to about 70% w/w, or from about 40% to about 60% w/w.

Formulations of the invention may comprise a low molecular weight polyethylene glycol, e.g., as a cosolvent. Low molecular weight polyethylene glycols may have an average molecular weight of between 200 and 10,000 AMU. For example, a formulation may comprise polyethylene glycol 400. In some embodiments, a formulation comprises one or more low molecular weight polyethylene glycols from about 0.5% to about 50% w/w, such as from about 1.0% to about 20% w/w, or from about 1.0% to about 10.0% w/w.

The formulations of the present invention may include propylene glycol as a cosolvent. If propylene glycol is used in the formulations, it can be from about 0.5% to about 20% w/w, such as from about 1.0% to about 15% w/w, or from about 2% to about 10% w/w.

In some embodiments, the formulations of the present invention contain from about 5% to about 20% w/w of cannabidiol, from about 0.01% to about 3.0% w/w of at least one sweetener, from about 0.01% to about 0.5% w/w of at least one antioxidant or stabilizing agent, from about 0.01% to about 1.0% w/w of at least one preservative, and a sufficient quantity of water and/or cosolvents.

In some embodiments, the formulations of the present invention contain from about 5% to about 15% w/w of cannabidiol, from about 0.01% to about 0.10% w/w of at least one sweetener, from about 0.03% to about 0.3% w/w of at least one antioxidant or stabilizing agent, from about 0.02% to about 0.06% w/w of at least one preservative, and from about 10% to about 80% w/w of at least one cosolvent.

In some embodiments, the composition comprises cannabidiol at a concentration of between about 5% and about 15% w/w. In some embodiments, the formulations of the present invention contain from about 8% to about 10% w/w of cannabidiol, from about 0.02% to about 0.08% w/w of at least one sweetener, from about 0.04% to about 0.20% w/w of at least one antioxidant or stabilizing agent, from about 0.02% to about 0.06% w/w of at least one preservative, and from about 30% to about 50% w/w of at least one cosolvent.

As used herein, “substantially pure” refers to a preparation having chromatographical purity of cannabidiol of greater than 98%, preferably greater than 98.5%, more preferably greater than 99.0%, and most preferably greater than 99.5%.

As used herein, “substantially devoid” as in “substantially devoid of THC” refers to a composition comprising cannabidiol having less than 0.3% w/w of THC (or cannabinoids other than cannabidiol) as determined by high-performance liquid chromatography (HPLC). Preferably, the composition contains less than 0.25% w/w of THC, such as less than 0.2% w/w, or even less than 0.1% w/w THC.

As used herein, all numerical values relating to amounts, weights, and the like, that are defined as “about” each particular value is plus or minus 10%. For example, the phrase “about 10% w/w” is to be understood as “9% w/w to 11% w/w.” Therefore, amounts within 10% of the claimed value are encompassed by the scope of the claims.

As used herein “% w/w” and “percent w/w” refers to the percent weight of the total composition or formulation.

As used herein the term “effective amount” refers to the amount necessary to treat a patient in need thereof.

As used herein the term “pharmaceutically acceptable” refers to ingredients that are not biologically or otherwise undesirable in an oral dosage form.

The term “preventing” is art-recognized, and when used in relation to a condition, such as a recurrence (e.g., a spasm or hypsarrhythmia), a disease, a syndrome complex, or any other medical condition, is well understood in the art, and includes administration of a composition to an asymptomatic subject which reduces the frequency or severity of, or delays the onset of, symptoms of a medical condition in the subject relative to a subject which does not receive the composition. Thus, prevention of infantile spasms includes, for example, reducing the number of spasms in a population of patients receiving a prophylactic treatment relative to an untreated control population, and/or delaying the manifestation of detectable spasms in a treated population versus an untreated control population, e.g., by a statistically and/or clinically significant amount. Prevention of infantile spasms also includes, for example, reducing the severity of a spasm experienced by subjects in a treated population versus an untreated control population.

As used herein, “qs” means a sufficient quantity of that component to reach a desired volume or concentration.

The disclosed embodiments are simply exemplary embodiments of the inventive concepts disclosed herein and should not be considered as limiting, unless the claims expressly state otherwise.

The following examples are intended to illustrate the present invention and to teach one of ordinary skill in the art how to use the formulations of the invention. They are not intended to be limiting in any way.

All claims, aspects, and embodiments of the invention, and specific examples thereof, are intended to encompass equivalents thereof.

### EXEMPLIFICATION

### Example 1

A composition comprising cannabidiol is prepared according to Table 1.

TABLE 1

| Formulation 1 | % w/w |
|---|---|
| Cannabidiol | 8.9 (80 mg/mL) |
| DL-α-Tocopherol | 0.05 |
| Ascorbyl Palmitate | 0.10 |
| Methyl Paraben | 0.02 |
| Propyl Paraben | 0.02 |
| Sucralose | 0.05 |
| Polyethylene Glycol 400 | 3.00 |
| Propylene Glycol | 7.50 |
| Purified Water | 30.00 |
| adjust pH to 6-7 (^{~}6.5) using sodium hydroxide (NaOH) | |
| Alcohol, qs to | 100.00 |

### Example 2

A composition comprising cannabidiol is prepared according to Table 2.

TABLE 2

| Formulation 2 | % w/w |
|---|---|
| Cannabidiol | 8.9 (80 mg/mL) |
| DL-α-Tocopherol | 0.05 |
| Ascorbyl Palmitate | 0.10 |
| Methyl Paraben | 0.02 |
| Propyl Paraben | 0.02 |
| Sucralose | 0.05 |
| Polyethylene Glycol 400 | 3.00 |
| Propylene Glycol | 7.50 |
| Purified Water | 30.00 |
| Alcohol, qs to | 100.00 |

### Example 3

A composition comprising cannabidiol is prepared according to Table 3.

TABLE 3

| Formulation 3 | % w/w |
|---|---|
| Cannabidiol | 8.9 (80 mg/mL) |
| DL-α-Tocopherol | 0.05 |
| Methyl Paraben | 0.02 |
| Propyl Paraben | 0.02 |
| Sucralose | 0.05 |
| Polyethylene Glycol 400 | 3.00 |
| Propylene Glycol | 7.50 |
| Purified Water | 30.00 |
| adjust pH to 6-7 (^{~}6.5) using NaOH | |
| Alcohol, qs to | 100.00 |

### Example 4

A composition comprising cannabidiol is prepared according to Table 4.

TABLE 4

| Formulation 4 | % w/w |
|---|---|
| Cannabidiol | 8.9 (80 mg/mL) |
| Butylated hydroxyanisole(BHA) | 0.05 |
| Sucralose | 0.05 |
| Methyl Paraben | 0.02 |
| Propyl Paraben | 0.02 |
| Polyethylene Glycol 400 | 3.00 |
| Water | 30.00 |
| adjust pH to 6-7 (^{~}6.5) using NaOH | |
| Alcohol, qs to | 100.00 |

### Example 5

Human subjects with video-EGG confirmed infantile spasms, between the ages of 6 months and 36 months, who have failed adequate treatments with hormonal therapy (adrenocorticotropic hormone (ACTH) (e.g., 150 IU/m2/day×2 weeks) and/or prednisolone (e.g., 8 mg/kg/day×2 weeks) and vigabatrin (e.g., 150 mg/kg/day×2 weeks)) will receive a composition comprising cannabidiol (“CBD”) according to the treatment schedule of Table 5. The control group receives placebo.

TABLE 5
CBD Titration Schedule

| | CBD Dosage |
|---|---|
| Protocol Days | (mg/kg/day ÷ TID*) |
| 1 | 5 |
| 2 | 10 |
| 3 | 20 |
| 4-14 | 40 |

*“TID” is ter in die, dosage administered three times a day

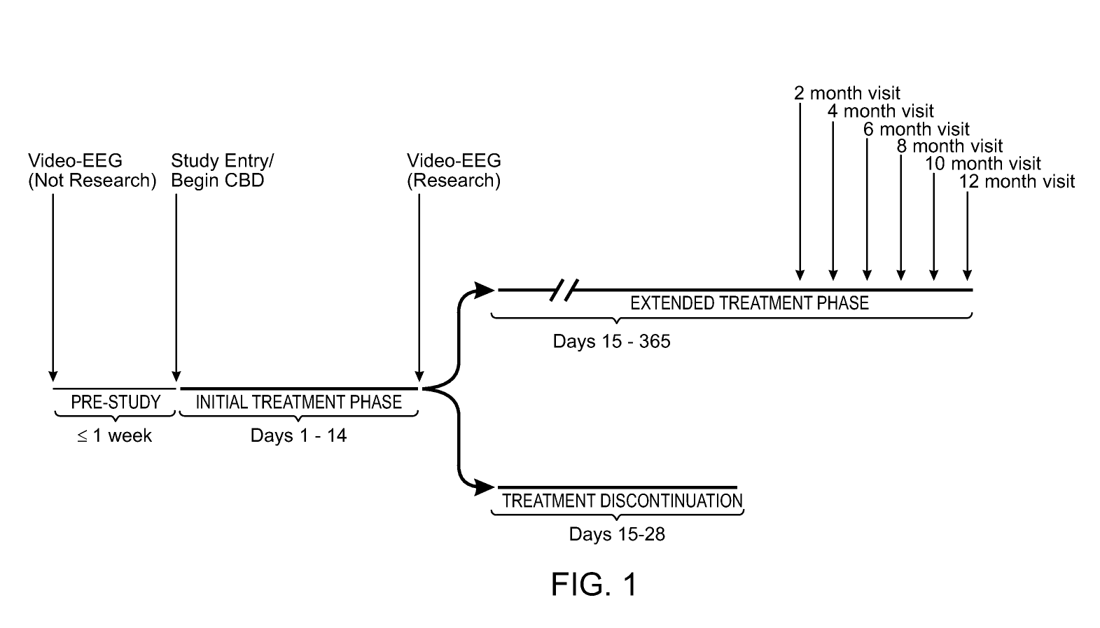

An overnight video-EEG is recorded on day 14, which shows reduced symptoms of infantile spasms in the cannabidiol group relative to the control group. The 40 mg/kg/day treatment is optionally extended for up to one year in the cannabidiol group for subjects who display a response (FIG. 1). The cannabidiol dosage is tapered in the cannabidiol group according to Table 6.

TABLE 6
CBD Taper Schedule

| | CBD Dosage |
|---|---|
| Protocol Days | (mg/kg/day ÷ TID) |
| 15-16 | 35 |
| 17-18 | 30 |
| 19-20 | 25 |
| 21-22 | 20 |
| 23-24 | 15 |
| 25-26 | 10 |
| 27-28 | 5 |
| 29 | Stop |

### Example 6

### Exemplary Treatment Regime

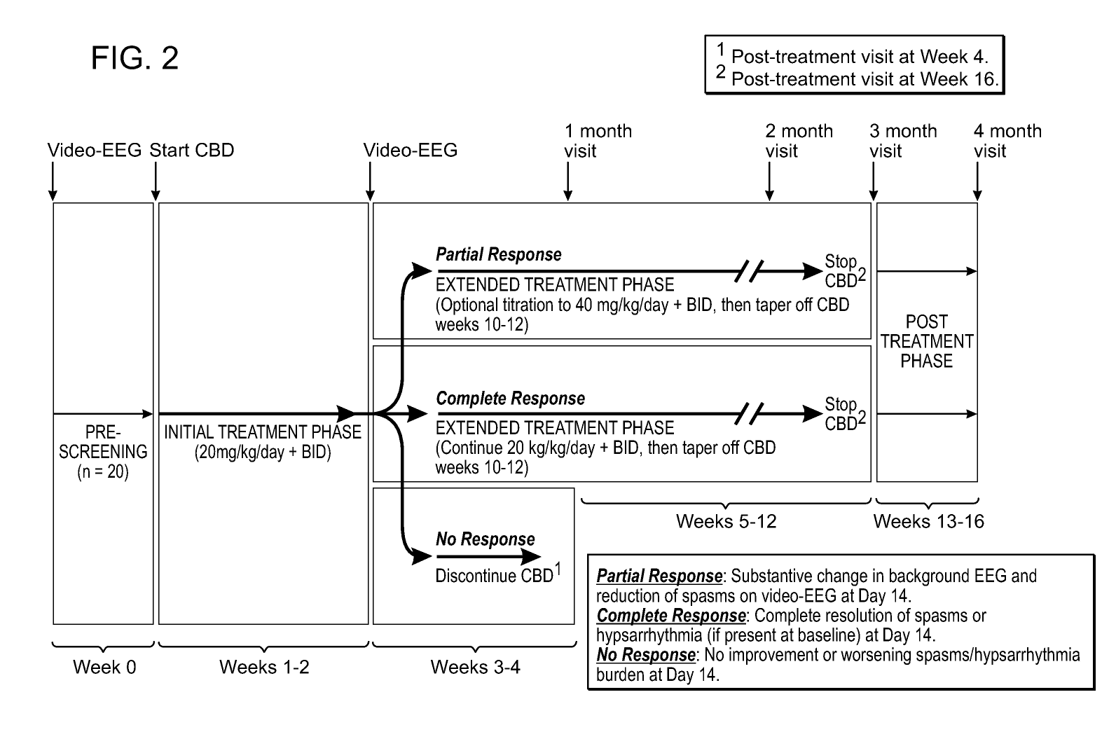

This is a study to evaluate the efficacy and safety of the composition comprising cannabidiol in the treatment of children aged 6 months through 36 months with a diagnosis of infantile spasms who have not responded to first line therapies. This study may comprise 4 periods: the Screening Period (up to 28 days), an Initial Treatment Period (2 weeks/14 Days), an Extended Treatment Period (10 weeks/70 Days), and a Follow-up Period (4 weeks/28 Days). The overall study duration is expected to be 20 weeks/140 Days for those subjects who respond to treatment with a composition comprising cannabidiol (FIG. 2).

Eligible subjects will complete all screening procedures within a maximum of 28 days before the Initial Treatment Period.

Video-EEG will be completed within the Screening Period and repeated at Day 14 (overnight video-EEG, minimum of 9 hours), following the Initial Treatment Period. Freedom from infantile spasms and hypsarrythmia will be evaluated. If there is clinical evidence of a complete response for patients on 40 mg/kg/day, video-EEG may be used for confirmation.

The first dose of the study drug (20 mg/kg/day [10 mg/kg BID]) (“BID” is bis in die, dosage administered twice a day) will be administered on Day 1 of the Initial Treatment Period. The investigator may conduct daily assessments either by telephone or email during the first 5 days of the Initial Treatment Period. The dose of study drug may be de-escalated at any time at the discretion of the investigator due to safety or tolerability concerns.

If subjects have a complete response to treatment, they may continue receiving the composition comprising cannabidiol at 20 mg/kg/day (10 mg/kg BID) up to Week 10/Day 70 (Extended Treatment Period), during which there will be monthly (±5 days) follow-up clinic visits. Subjects will be tapered off study drug over a 2-week period (Weeks 10-12/Days 70-84). A recommended tapering regimen is as follows: 80% of the dose for 2 days, 60% for 2 days, 40% for 3 days, 20% for 3 days, 10% for 4 days and then off but can be adjusted based on patient's response. There will be a post-treatment visit at week 16/Day112. Overall study duration will be up to 17 weeks for complete responders. Patients' response to treatment is categorized according to Table 7.

If, at Day 14, subjects have a partial response to treatment, they may continue receiving composition comprising cannabidiol titrating up to 40 mg/kg/day (20 mg/kg BID) up to Week 10 (Extended Treatment Period), during which there will be monthly (±5 days) follow-up clinic visits. Recommended titration is as follows: 25 mg/kg/day for 5 days, 30 mg/kg/day for 5 days, 35 mg/kg/day for 5 days, and 40 mg/kg/day but can be adjusted by the Investigator based on patients' response. The Investigator will conduct telephone and/or email assessments of subjects every 5 days, or as deemed necessary, during this treatment period. The dose of composition comprising cannabidiol may be de-escalated at any time at the discretion of the Investigator due to safety or tolerability concerns. Subjects will be tapered off study drug over a 2-week period (Weeks 10-12). Recommended tapering regimen: 80% of the dose for 2 days, 60% for 2 days, 40% for 3 days, 20% for 3 days, 10% for 4 days and then off but can be adjusted based on patient's response. There will be a post-treatment visit at Week 16 (Visit 7). If there is clinical evidence of increased response, video-EEG may be used for confirmation. Treatment with composition comprising cannabidiol will be continued until confirmed by an independent video-EEG reader. Overall study duration will be up to 20 weeks/160 Days for Partial Responders.

If no response is observed at Day 14, study drug will be discontinued. Overall study duration will be up to 8 weeks/56 Days, including an up to 28-day Screening Period and a 2-Week Initial Treatment Period. A follow-up visit will be completed on week 4/Day 28.

TABLE 7
Day 14 Response Categories

| Response Category | Definition |
|---|---|
| Complete Response | Complete resolution of spasms and |
| | hypsarrythmia (if present at baseline) |
| | confirmed by video-EEG at Day 14 |
| Partial Response | Substantive change in background EEG |
| | or reduction in spasms on video-EEG |
| | obtained at Day 14 |
| Non Response | No improvement or worsening of |
| | spasms/hypsarrythmia burden at Day 14 |

We claim:

1. A method of treating or preventing infantile spasms, hypsarrhythmia, lightning attacks, nodding attacks, salaam attacks, and/or jackknife attacks in a subject, comprising administering to the subject a composition comprising cannabidiol (“CBD”).

2. The method of claim 1, wherein the composition comprises tetrahydrocannabinol (“THC”) at a concentration of less than 0.3% w/w.

3. The method of claim 1, wherein the composition is substantially devoid of cannabinoids selected from cannabichromene, cannabichromevarin, cannabicitran, cannabicyclol, cannabidivarin, cannabielsoin, cannabigerol, cannabigerol monomethyl ether, cannabigerovarin, cannabinol, cannabivarin, iso-tetrahydrocannabinol, and tetrahydrocannabivarin.

4. The method of claim 3, wherein the composition comprises cannabinoids selected from cannabichromene, cannabichromevarin, cannabicitran, cannabicyclol, cannabidivarin, cannabielsoin, cannabigerol, cannabigerol monomethyl ether, cannabigerovarin, cannabinol, cannabivarin, iso-tetrahydrocannabinol, and tetrahydrocannabivarin at a concentration of less than 0.3% w/w.

5. The method of claim 1, wherein the composition comprises cannabidiol at a concentration of between about 2% and about 50% w/w.

6. The method of claim 5, wherein the composition comprises cannabidiol at a concentration of between about 5% and about 15% w/w.

7. The method of claim 1, wherein between about 5 mg/kg and about 100 mg/kg of cannabidiol is administered to the subject per day.

8. The method of claim 7, wherein 20 mg/kg of cannabidiol is administered to the subject per day.

9. The method of claim 8, further comprising, after an initial treatment period, increasing the dose of cannabidiol administered to the patient, e.g., from 20 mg/kg to 40 mg/kg per day, if the patient experienced a spasm during the initial treatment period.

10. The method of claim 7, wherein about 40 mg/kg of cannabidiol is administered to the subject per day.

11. The method of claim 1, wherein less than 1 mg of THC is administered to the subject per day.

12. The method of claim 1, wherein less than 1 mg of cannabinoids selected from cannabichromene, cannabichromevarin, cannabicitran, cannabicyclol, cannabidivarin, cannabielsoin, cannabigerol, cannabigerol monomethyl ether, cannabigerovarin, cannabinol, cannabivarin, iso-tetrahydrocannabinol, and tetrahydrocannabivarin is administered to the subject per day.

13. The method of claim 1, wherein the composition is administered 1, 2, 3, 4, or 5 times per day.

14. The method of claim 1, wherein the composition comprising cannabidiol is administered to the subject for a period of about 12 weeks.

15. The method of claim 1, wherein the subject is not responsive to adrenocorticotropic hormone therapy, prednisone or prednisolone therapy, and/or vigabatrin therapy.

16. The method of claim 1, wherein the subject is responsive to adrenocorticotropic hormone therapy, prednisone or prednisolone therapy, and/or vigabatrin therapy.

17. The method of any one of claim 1, wherein the subject is human.

18. The method of claim 17, wherein the subject is between 1 week old and 18 years old.

19. The method of claim 1, wherein the composition comprising CBD is substantially devoid of tetrahydrocannabinol (“THC”).

20. A method of treating infantile spasms in a subject, comprising administering cannabidiol (“CBD”) to a subject.
